(19) United States
(12) Patent Application Publication
(10) Pub. No.: US 2005/0107871 A1
(43) Pub. Date: May 19, 2005

(54) Apparatus and methods for valve repair

(76) Inventors: Fidel Realyvasquez, Palo Cedro, CA (US); Laurent Schaller, Los Altos, CA (US)

Correspondence Address: Jeffrey J. Hohenshell, 710 Medtronic Parkway, Minneapolis, MN, 55432, US

(21) Appl. No.: 10/814,854

(22) Filed: Mar. 30, 2004

Related U.S. Application Data
(60) Provisional application No. 60/459,385, filed on Mar. 30, 2003.

(51) Int. Cl. A61F 2/24; A61B 17/128

(52) U.S. Cl. 623/2.11; 623/2.17; 606/142

(57) ABSTRACT

A valve implant or prosthesis includes a skirt or prosthetic valve leaflet configured to cover one of the leaflets of the valve to be repaired in a patient's heart. In one embodiment, a heart valve prosthesis includes a curved member and a skirt. The curved member can have first and second ends and be adapted to form a partial ring along a portion of one of the valve annulae in the patient's heart. Alternatively, the curved member can form a full ring that is adapted to extend along the entire valve annulus. The skirt extends along the curved member and depends therefrom. This prosthesis is especially useful in treating mitral valve insufficiency. In this case, the skirt can be configured so that when the prosthesis is secured to the mitral valve along the mitral valve annulus, the skirt covers the posterior leaflet and the opposed edges of the skirt and the anterior leaflet coapt. In addition, when the curved member is secured to the posterior portion of the mitral valve annulus, further annulus dilation can be minimized or eliminated. Implant delivery apparatus is provided for rapid implant delivery and securement to the valve.

### CROSS-REFERENCE

This application claims the benefit of U.S. Provisional Application No. 60/459,385, filed Mar. 30, 2003 and entitled Apparatus and Methods for Valve Repair, which application is incorporated herein by reference.

### FIELD OF THE INVENTION

The invention relates to heart valve repair and particularly to valve leaflet replacement. The invention is especially useful in mitral valve repair procedures, which generally involve correction of mitral insufficiency (e.g., mitral valve regurgitation when the mitral valve does not properly close).

### BACKGROUND OF THE INVENTION

Essential to normal heart function are four heart valves, which allow blood to pass through the four chambers of the heart in one direction. The valves have either two or three cusps, flaps, or leaflets, which comprise fibrous tissue that attaches to the walls of the heart. The cusps open when the blood flow is flowing correctly and then close to form a tight seal to prevent backflow.

The four chambers are known as the right and left atria (upper chambers) and right and left ventricles (lower chambers). The four valves that control blood flow are known as the tricuspid, mitral, pulmonary, and aortic valves. In a normally functioning heart, the tricuspid valve allows one-way flow of deoxygenated blood from the right upper chamber (right atrium) to the right lower chamber (right ventricle). When the right ventricle contracts, the pulmonary valve allows one-way blood flow from the right ventricle to the pulmonary artery, which carries the deoxygenated blood to the lungs. The mitral valve, also a one-way valve, allows oxygenated blood, which has returned to the left upper chamber (left atrium), to flow to the left lower chamber (left ventricle). When the left ventricle contracts, the oxygenated blood is pumped through the aortic valve to the aorta.

Certain heart abnormalities result from heart valve defects, such as valvular insufficiency. For example, mitral valve insufficiency, also known as mitral regurgitation, is a common cardiac abnormality where the mitral valve leaflets do not completely close when the left ventricle contracts. This allows blood to flow back into the left atrium, which then requires the heart to work harder as it must pump both the regular volume of blood and the blood that has regurgitated back into the left atrium. If this insufficiency is not corrected, the added workload can eventually result in heart failure.

Various approaches to correct valve defects have included valve replacement, valve leaflet repair, chordae tendineae shortening or replacement, and or valve annulus repair also known as annuloplasty. One example where annuloplasty procedures have been developed is in the field of mitral valve insufficiency correction.

Mitral valve insufficiency typically results from a change in the size and shape of the mitral valve annulus. Mitral valve annuloplasty involves reestablishing the normal shape and size of the mitral valve annulus so that it can effect full closure of the valve leaflets.

Approaches to improve valve function (e.g., mitral or tricuspid valve function) have included tissue plication devices and reinforcement of the valve annulus with annuloplasty rings. These approaches have been stated to reestablish the original annulus size and shape and/or prevent further annulus dilation.

Both rigid and flexible annuloplasty rings have been developed. Rigid rings, which generally tend to dictate the shape and contour of the mitral valve annulus, have been considered to somewhat compromise the natural flexibility of the annulus. Flexible annuloplasty rings emerged to provide some degree of compliance in the valve annulus so that the valve could maintain normal physiological motion throughout the cardiac cycle of a beating heart. This is in addition to providing annulus reinforcement. However, it is believed that among the drawbacks of these rings is that they may fold or crimp during implantation and thereby undesirably reduce the size of the valve (e.g., the mitral valve) opening. Also, the sutures used to secure the ring may cause scarring and stiffening of the valve annulus and reduce annulus flexibility over time.

C-shaped bands or partial annuloplasty rings also have been developed. These devices can be attached solely to the posterior portion of the valve annulus which eliminates the need to attach material to the anterior portion of the annulus. Full and partial ring devices are disclosed, for example, in U.S. Pat. No. 3,656,185, which issued to Carpentier.

Other attempts to improve upon valve repair procedures include those described in U.S. Pat. No. 5,450,860, which issued to O'Connor, U.S. Pat. No. 6,183,512, which issued to Howanec, Jr. et al., and U.S. Pat. No. 6,250,308, which issued to Cox.

The O'Connor patent discloses a plication approach, particularly suitable for use with an annuloplasty operation on heart valves (e.g., mitral or tricuspid valves). The approach involves a ligament, which can comprise a wide, flexible strip of expanded polytetrafluorethylene or similar material, and sutures to retain the ligament in place. The ligament has at least an end of constricted diameter and a needle attached thereto, or it can have two constricted ends and a needle attached to each of the ends. This construction permits the ligament to be drawn through an area of tissue to be plicated. Once in place, a first end of the ligament is anchored, preferably with sewing of conventional sutures through the ligament, and the tissue is cinched along the length of the ligament to provide the desired amount of plication. Once the tissue is correctly oriented, the second end of the ligament is then likewise anchored in place, again preferably through the use of a suture sewn through the ligament.

The Howanec patent describes a system that includes an elongate flexible band with a needle attached to one end of the band and a fit adjuster attached to the other end of the band. The needle is used to introduce the band into the atrioventricular groove (hereafter “AV groove”) and then pull a portion of the band out of the tissue. After the band is so implanted into the AV groove, a fit adjuster is used to couple the exposed ends of the band and size and position the band in the annulus. The band is positioned and sized by pulling it through the fit adjuster and cinching the tissue in the AV groove until the valve annulus is reconfigured to an optimal shape. The band can be secured to the valve annulus with sutures and the exposed portions of the annuloplasty system removed.

The Cox patent describes a system that comprises a combined annuloplasty ring implant, which has a rigid section and a flexible section. A needle is coupled to one end of the implant. The needle facilitates introducing the implant into the fatty pad of the AV groove, which surrounds the valve annulus, at one end of the posterior portion of the annulus and pulling one end portion of the implant out of the AV groove in the vicinity of the other end of the posterior portion of the annulus. The flexible section of the ring extends adjacent to the flexible posterior portion of the annulus, while the rigid section of the ring spans the substantially rigid inter-trigone section of the annulus. Cox advances that with this procedure one need not suture the flexible section directly to the mitral valve annulus, thereby substantially eliminating scarring and stiffening of the annulus. In one example, the flexible material is also elastic to accommodate the expansion and contraction of the annulus, in addition to flexing. The system further includes means for joining the ends of the ring, which are positioned along the inter-trigone section, after the needle is removed. Sutures can be added to secure the annuloplasty ring to the annulus, for example, along the inter-trigone section.

Other plication and valve repair approaches are disclosed in PCT International Patent Application Nos. PCT/US01/42653 and PCT/US01/31709, which were published under publication numbers WO 02/30298 and WO 02/30295 and entitled “Minimally Invasive Annuloplasty Procedure and Apparatus” and “Minimally Invasive Valve Repair Procedure and Apparatus,” respectively, and U.S. Patent Application Publication No. US-2003-0074012 entitled “Minimally Invasive Annuloplasty Procedure and Apparatus.” These approaches, in-part, address various inherent disadvantages with prior open heart surgical procedures as described, for example, by F. Maisano, et al. in their article entitled “The double-orifice technique as a standardized approach to treat mitral regurgitation due to severe myxomatous disease” which appeared in European Journal of Cardio-thoracic Surgery, Vol. 17 (2000) 201-205. Disadvantages associated with such open-heart procedures include cumbersome suture management, timely knot tying steps, pain, and long recovery time.

Applicants believe that there remains a need for improved valvular repair apparatus and methods.

### SUMMARY OF THE INVENTION

The present invention involves valve repair apparatus and methods that overcome problems and disadvantages of the prior art. According to one aspect of the invention, a valve implant or prosthesis is provided, which includes a skirt or prosthetic valve leaflet that is configured to cover and/or replace one of the leaflets of the valve and a member or mechanism for holding the leaflet in place. Other aspects of the invention include, but are not limited to, heart valve repair apparatus for delivering heart valve prosthesis to a target site and a method for delivering heart valve prosthesis.

In one embodiment of the invention, heart valve prosthesis includes a curved member and a skirt. The curved member can have first and second ends and be adapted to form a partial ring along a portion of one of the valve annulae in the patient's heart. Alternatively, the curved member can form a full ring that is adapted to extend along the entire valve annulus. The skirt extends along the curved member and depends therefrom. This prosthesis is especially useful in treating mitral valve insufficiency. In this case, the skirt can be configured so that when the prosthesis is secured to the mitral valve along the mitral valve annulus, the skirt covers the posterior leaflet and the opposed edges of the skirt and anterior leaflet coapt. In addition, when the curved member is secured to the posterior portion of the mitral valve annulus, further annulus dilation can be minimized or eliminated.

According to another embodiment of the invention, heart valve delivery apparatus for placing heart valve prosthesis in a patient's heart comprises a delivery device comprising a plurality of tube pairs arranged to support the heart valve prosthesis; and a plurality of self-closing clips, each clip having an open configuration and a closed configuration and first and second piercing ends, each clip being ejectably mounted to one of the tube pairs with a first portion of the clip slidably positioned in one tube of the tube pair and a second portion slidably positioned in the other tube of the tube pair so that the first clip piercing end can be ejected from the one tube of the tube pair and the second piercing end can be ejected from the other tube of the tube pair.

According to another embodiment of the invention, heart valve repair apparatus for placing heart valve prosthesis in a patient's heart comprises heart valve prosthesis comprising a prosthetic valve leaflet and a member supporting the leaflet; delivery apparatus comprising a support for the valve prosthesis and a plurality of clips ejectably mounted to the delivery apparatus support, each clip having two piercing tips extending into the member supporting the leaflet.

According to another embodiment of the invention, a method for delivering heart valve prosthesis comprises providing heart valve prosthesis having a curved member and a skirt extending therefrom and a plurality of self-closing clips, each having two pointed ends, and an open configuration and a closed configuration; securing the curved member to said plurality of self-closing clips with the two pointed ends of each clip penetrated into the curved member; placing the curved member on the mitral valve annulus of a patient's heart; ejecting all of the clips simultaneously to penetrate into the valve annulus and move toward their closed configuration to secure the heart valve prosthesis to the valve annulus.

The above is a brief description of some deficiencies in the prior art and advantages of the present invention. Other features, advantages, and embodiments of the invention will be apparent to those skilled in the art from the following description and accompanying drawings, wherein, for purposes of illustration only, specific forms of the invention are set forth in detail.

### BRIEF DESCRIPTION OF THE DRAWINGS

FIG. 1A is a perspective view of one embodiment of a valve prosthesis in accordance with the principles of the present invention with the prosthetic leaflet in a closed position;

FIG. 1B is a perspective view of the valve prosthesis of FIG. 1A with the prosthetic leaflet in an open position;

FIG. 2A is a top plan view of the valve prosthesis of FIG. 1A;

FIG. 2B is a top plan view of the valve prosthesis of FIG. 1B;

FIG. 3A is a side elevational view of the prosthesis of FIG. 1A;

FIG. 3B is a side elevational view of the prosthesis of FIG. 1B;

FIGS. 4A-C are partial sectional views of a clip delivery mechanism for securing the prosthesis of FIG. 1A to a patient's valve where FIG. 4A depicts the clip in a first loaded position, FIG. 4B depicts the clip in an intermediate position, and FIG. 4C depicts the clip ejected from the delivery mechanism;

FIGS. 5A-C are longitudinal partial cross sections of the clip delivery mechanism of FIGS. 4A-C where FIG. 5A depicts the clip in a first loaded position, FIG. 5B depicts the clip in an intermediate position, and FIG. 5C depicts the clip ejected from the delivery mechanism;

FIGS. 6A-C are partial cross sections of the clip delivery mechanism of FIGS. 5A-C rotated 90 degrees where FIG. 6A is taken along line 6A-6A of FIG. 5A illustrating the clip in a first loaded position, FIG. 6B depicts the clip of FIG. 6A in an intermediate position, and FIG. 6C depicts the clip of FIG. 6A ejected from the delivery mechanism;

FIG. 7 is a perspective view of prosthesis delivery apparatus, which in the illustrative embodiment, includes a plurality of the delivery mechanisms of FIG. 4A-C;

FIGS. 8A-8E illustrate delivery and securement of the prosthesis of FIG. 1 using the prosthesis delivery mechanism of FIG. 7 where FIG. 8A is a perspective view of the prosthesis delivery apparatus of FIG. 7 and the prosthesis of FIG. 1A secured thereto and positioned for securement to a mitral valve annulus, FIG. 8B illustrates the prosthesis delivery mechanism of FIG. 7 seated on the valve annulus; FIG. 8C illustrates simultaneous ejection of all of the clips from the clip delivery mechanisms with a single actuation mechanism, FIG. 8D illustrates the clips securing the valve prosthesis in place along the valve annulus and removal of the prosthesis delivery apparatus, and FIG. 8E illustrates a top view of the valve prosthesis in place over the mitral valve with the anterior leaflet in view and in a closed position and with the prosthetic leaflet or skirt covering the posterior leaflet.

### DETAILED DESCRIPTION OF THE INVENTION

Before the present invention is described, it is to be understood that this invention is not limited to the particular embodiments or examples described herein, as such may, of course, vary. Further, when referring to the drawings, like numerals indicate like elements.

According to one aspect of the invention, a valve implant or prosthesis includes a skirt or prosthetic valve leaflet that is configured to cover and/or replace one of the leaflets of the valve and a member or mechanism for holding the leaflet in place.

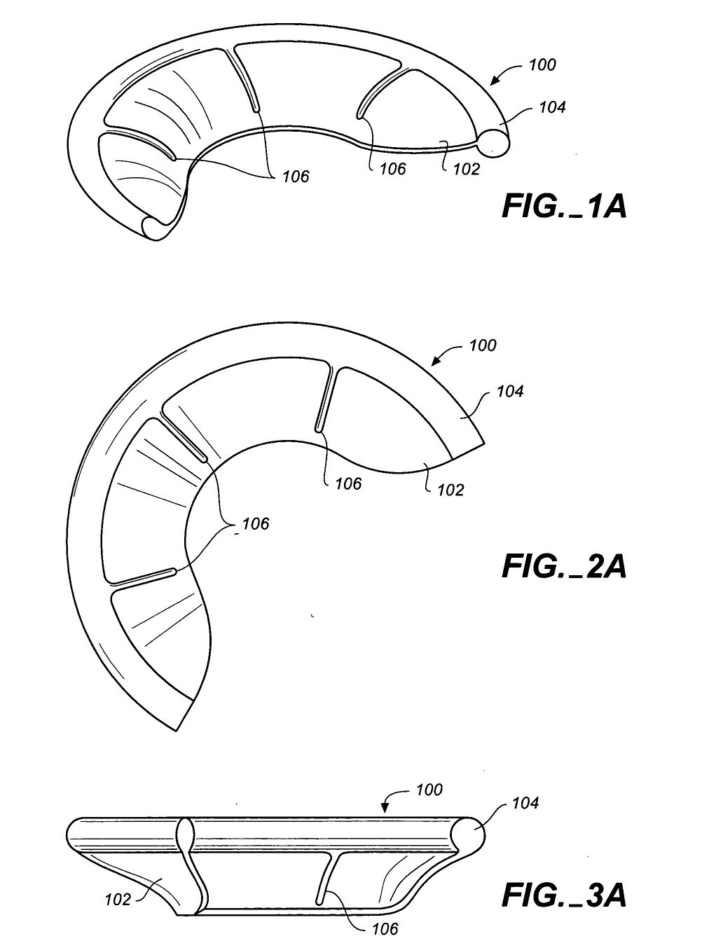

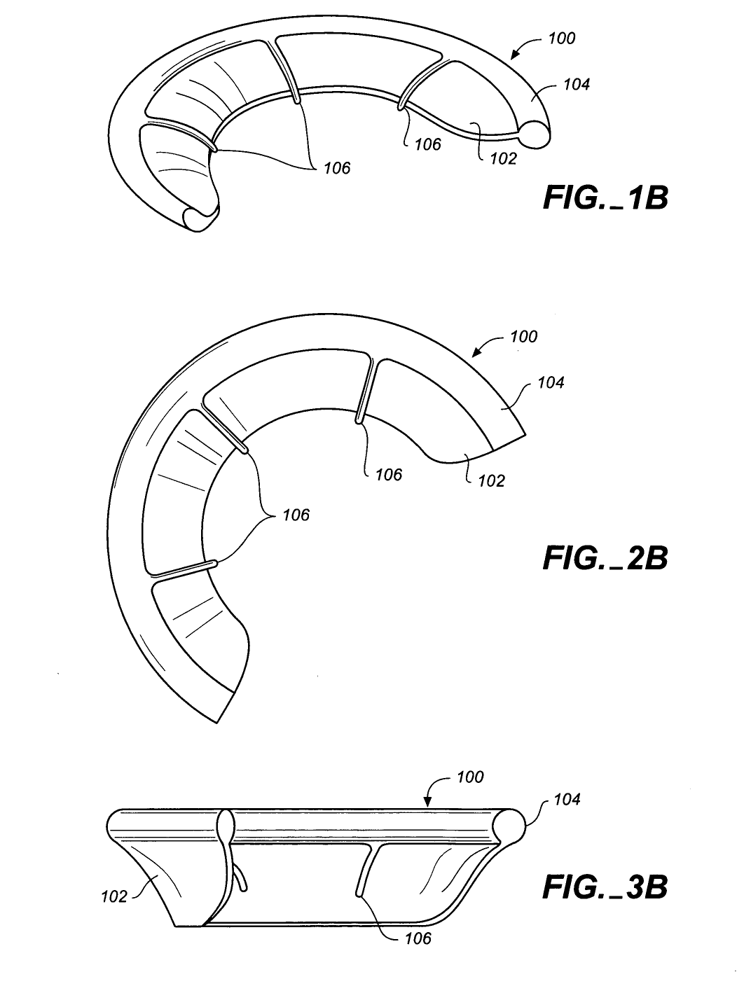

Referring to FIGS. 1A and B, 2A and B and 3A and B, plan and side view of one embodiment of a valve prosthesis, generally designated with reference numeral 100 and including a replacement valve leaflet is shown in accordance with the principles of the present invention. The replacement valve leaflet is shown in a closed configuration in FIGS. 1A, 2A, and 3A, and in an open configuration in FIGS. 1B, 2B, and 3B.

Referring to 1A, 2A and 3A, exemplary valve prosthesis 100 includes a skirt or prosthetic leaflet 102, which is configured to replace or extend over and cover a leaflet in the valve under repair (e.g., the mitral valve posterior leaflet). Skirt or valve leaflet 102 can, for example, be made from ePTFE or prosthetic tissue. One prosthetic tissue that can be used is pig leaflet tissue. When repairing a mitral valve, the skirt can be configured to cover the posterior leaflet and effectively replace the posterior leaflet without removing it.

Skirt 102 is secured to a member or mechanism for holding it in the desired location. In the illustrative embodiment, skirt 102 is secured to curved member 104, which can be in the form of an open or partial annuloplasty ring. Skirt 102 can be secured to ring 104 by gluing, using conventional medical gluing materials, or sewing or it can be wrapped around ring 104 and glued or fused to itself. Although not shown, it should be understood that the curved member also can be in the form of a full, continuous or closed annuloplasty ring.

Member 104 can be made from any suitable material(s) such as from one or more biocompatible polymers including but not limited to silicone. It also can be covered with Dacron® material such as synthetic polyester textile fiber material or fibrous mesh to assist with tissue ingrowth after implantation. Further, curved member 104 can be rigid or flexible. Rigid or nonpliable rings, whether full or partial, can improve the ability to reshape the mitral valve annulus. Flexible rings, whether full or partial, can more readily conform to the mitral valve annulus and accommodate valve movement. In the case where curved member 104 is to be rigid or nonpliable, suitable plastics can be used. Alternatively, it can be reinforced with a stainless steel or titanium insert(s), which can be in the form of threads or wires extending generally parallel to the longitudinal axis of the curved member, e.g., curved member 104.

Curved member 104 also can be provided with a plurality of struts 106 that extend radially therefrom in an inward direction and provide reinforcement or support for skirt 102. More specifically, the struts can be curved radially inward and downward to conform to the surface or curvature of replacement leaflet 102 when replacement leaflet 102 is in its desired closed position during diastole. The struts, which can be made from the same material as member 104, can be attached to curved member 104 or integrally formed therewith, but are not attached to skirt 102 so that the skirt can move away form the struts during diastole and toward or to the struts during systole. Since the replacement valve leaflet does not have chordae tendineae, the struts are provided to prevent the replacement valve leaflet from folding backward during the systolic cycle. The struts, however, do not extend completely to the inner perimeter of skirt 102 (see e.g., FIG. 1A). The inner circumferential margin of the skirt that extends inwardly beyond the struts facilitates contact or apposition between the skirt and the opposed leaflet to effect a seal therebetween during systole. Otherwise, one or more of the struts may contact the opposed leaflet and form a gap and cause regurgitation. The inner circumferential margin can range from about 1 to 3 mm.

The prosthesis can be secured to the valve by suturing or the use of clips or other fasteners. It can simply be placed on the desired location of the valve and the fasteners placed to secure the prosthesis to the valve. Examples of suitable clips are described in, but not limited to, U.S. Pat. No. 5,972,024 to Northrup, et al. and entitled “Suture-Staple Apparatus and Method,” U.S. Pat. No. 6,514,265 to Ho, et al. and entitled “Tissue Connector Apparatus with Cable Release,” and U.S. Pat. No. 6,613,059 to Schaller, et al. and entitled “Tissue Connector Apparatus and Methods,” the disclosures of which are hereby incorporated herein by reference. Alternatively, the prosthesis can be more rapidly secured to the valve using clip delivery apparatus and/or valve prosthesis delivery apparatus constructed according to further aspects of the invention.

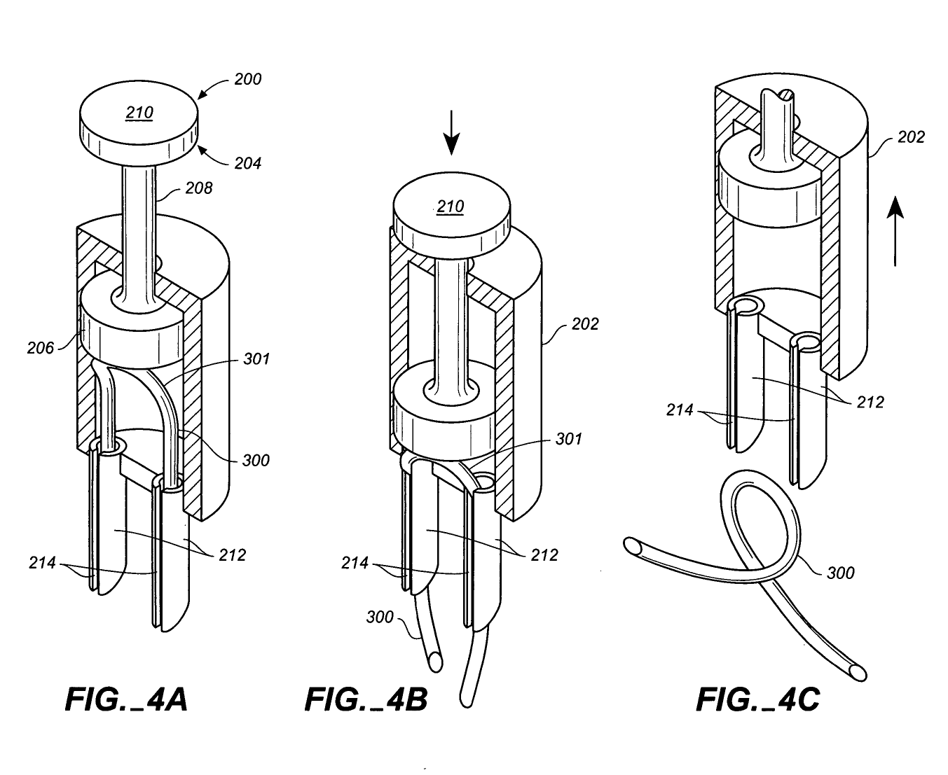

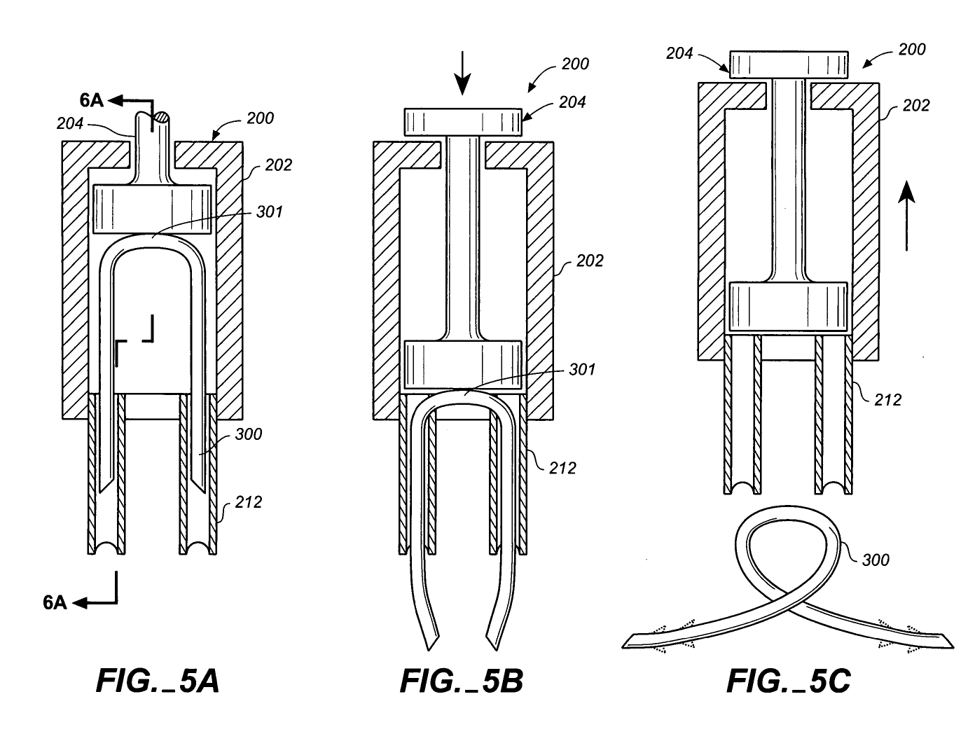

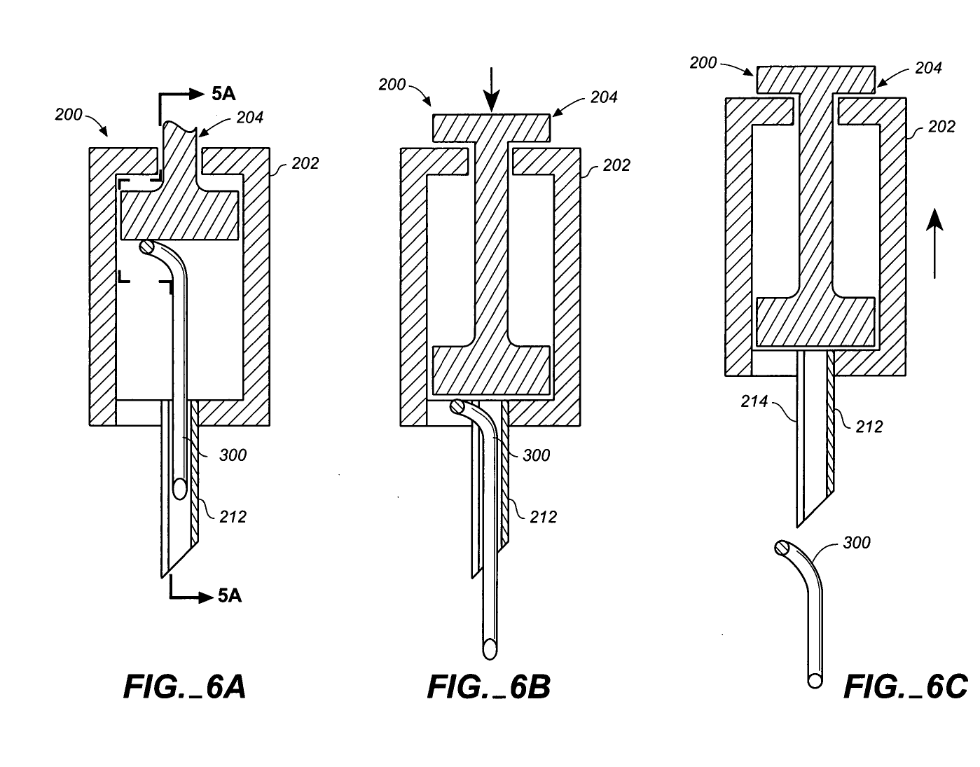

FIGS. 4A-C are partial sectional views of one exemplary embodiment of clip delivery apparatus, which is generally designated with reference numeral 200, for ejecting fasteners through the prosthesis and securing the prosthesis a patient's valve. Apparatus or mechanism 200 includes a cylindrical housing 202 and an ejector or plunger 204 slidably mounted therein. Plunger 204 includes a piston head 206 and a piston rod 208 extending therefrom and terminating in an actuator member or anvil 210. Clip delivery apparatus 200 further includes fastener guide tubes 212, which can be hypotubes and which can have longitudinal slots 214 extending therethrough. Each guide tube can be integrally formed with housing 202 or they may be separately formed and secured to the housing by gluing or welding. Referring to FIGS. 4A-C, 5A-C, and 6A-C, as the anvil is pressed and the piston nears or contacts the guide tubes, the self-closing clip shown in the drawings is ejected and if unrestrained, returns to its relaxed state as shown in FIGS. 4C, 5C, and 6C. Specifically, when each clip is restrained in a respective guide tube 212, the upper end of each clip 300, is angulated forward and is outside the guide tubes as shown, for example, in FIGS. 4A-4B and 5A-B. This angulated portion of the clip, which also joins the illustrated generally straight clip portions, is designated with reference numeral 301. As piston head 206 is pushed distally, it pushes angulated portion 301, which then pulls the portions adjacent thereto therewith and out of slots 214 (see e.g., FIG. 5B). Once those portions of the clip begin to come out through slots 214, the remainder of the clip follows because the clip is spring loaded in the tubes and wants to return to its memory shape or free state.

One fastener that can be used with clip delivery apparatus is a self-closing clip. One such clip is shown in its open, deformed configuration in FIG. 4A and in a relaxed, free state or closed configuration in FIG. 4C. The illustrative clip of FIG. 4C can be described as having a closed loop configuration. The clip is generally designated with reference numeral 300. Clip 300 has pointed or sharpened ends for piercing through curved member 104 and the valve annulus as will be described in more detail below. Further, clip 300 can have barbs as shown in dashed line in FIG. 5C to enhance securement of the prosthesis to the valve annulus.

The clip can comprise wire made from shape memory alloy or elastic material so that it tends to return to its memory shape after being released from the clip delivery apparatus. As is well known in the art, shape memory material has thermal or stress relieved properties that enable it to return to a memory shape. For example, when stress is applied to shape memory alloy material causing at least a portion of the material to be in its martensitic form, it will retain its new shape until the stress is relieved as described in U.S. Pat. No. 6,514,265 to Ho, et al. and entitled “Tissue Connector Apparatus with Cable Release” and U.S. Pat. No. 6,641,593 to Schaller, et al. and entitled “Tissue Connector Apparatus and Methods,” the disclosures of which are hereby incorporated herein by reference. Then, it returns to its original, memory shape. Accordingly, at least a portion of the shape memory alloy of clip 300 is converted from its austenitic phase to its martensitic phase when the wire is in its deformed, open configuration (see e.g., FIG. 4A). As the stress is removed, the material undergoes a martensitic to austenitic conversion and springs back to its undeformed configuration (see e.g., FIG. 4C). One suitable shape memory material for the clip 300 is a nickel titanium (nitinol) based alloy, which exhibits such pseudoelastic (superelastic) behavior.

The nitinol may include additional elements which affect the yield strength of the material or the temperature at which particular pseudoelastic or shape transformation characteristics occur. The transformation temperature may be defined as the temperature at which a shape memory alloy finishes transforming from martensite to austenite upon heating (i.e., A_{f} temperature). The shape memory alloy preferably exhibits pseudoelastic (superelastic) behavior when deformed at a temperature slightly above its transformation temperature. As the stress is removed, the material undergoes a martensitic to austenitic conversion and springs back to its original undeformed configuration. In order for the pseudoelastic wire to retain sufficient compression force in its undeformed configuration, the wire should not be stressed past its yield point in it deformed configuration to allow complete recovery of the wire to its undeformed configuration. The shape memory alloy is preferably selected with a transformation temperature suitable for use with a stopped heart condition where cold cardioplegia has been injected for temporary paralysis of the heart tissue (e.g., temperatures as low as 9-10 degrees Celsius).

The clip can be made by wrapping a nitinol wire having a diameter in the range of about 0.002 to 0.015 inch, and preferably 0.011 inch, and wrapping it around a mandrel having a diameter in the range of about 0.050 to 0.150 inch, and preferably 0.100 inch. The heat treatment of the nitinol wire to permanently set its shape as shown in FIG. 4C can be achieved by heat-treating the wire and mandrel in either a convection oven or bath at a temperature range of 400 to 600° C., preferably 450° C., for a duration of about 1 to 45 minutes, preferably 15 minutes.

According to another aspect of the invention, valve prosthesis delivery apparatus is provided to rapidly deliver the valve prosthesis to the surgical site and to secure the prosthesis at the desired location.

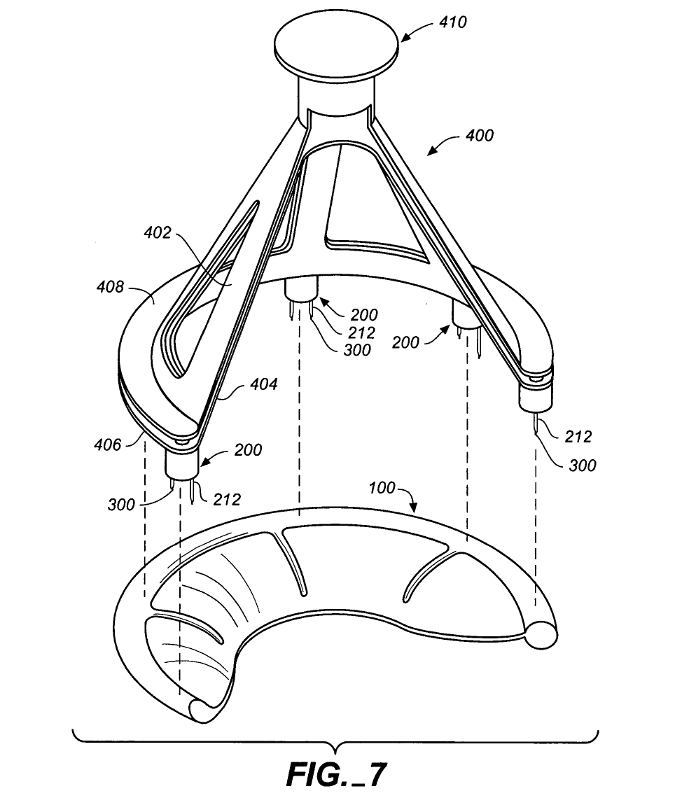

Referring to FIG. 7, an exemplary embodiment of a valve prosthesis delivery mechanism, which is generally designated with reference numeral 400, is shown. Valve prosthesis delivery apparatus 400 includes a first member 402 slidably or movable coupled to a second member 404. Members 402 and 404 are shown as being in a frustoconical shape with cut outs to enhance visibility of the surgical site and lighten the apparatus. Members 402 and 404 also are configured so that member 404 fits within member 402. In the example provided in FIG. 7, member 404 is nested in member 402. Alternatively speaking, member 402 is stacked on member 404.

Second member 404 includes a clip delivery support(s) for supporting a plurality of clip delivery devices 200. In the illustrative embodiment, a clip delivery support is shown in the form of a partial flat ring 406. Ring 406 has a plurality of holes formed therein in which piston rods 208 of clip delivery apparatus 200 or devices are disposed. First member 402 includes a head(s) or anvil(s) adapted to push clip ejectors 204 in a distal direction to eject clips 300. In the illustrative embodiment, a first member head or anvil is shown in the form of a partial flat ring 408. First member 402 also includes a plunger knob or grip 410 to push member 402 downwardly when the prosthesis delivery apparatus is positioned over the surgical site as will be discussed in more detail below. Grip 410 can be in the form of a cylinder with a cap at one end (a closed end cylinder) extending from the frustoconical body portion of first member 402 as shown in FIG. 7.

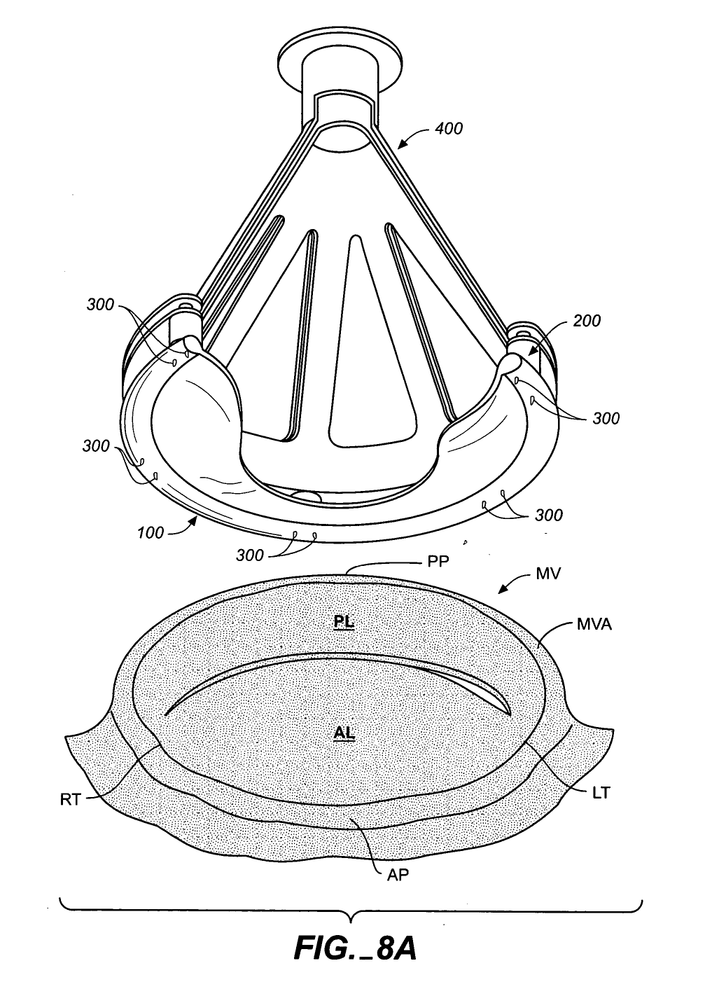

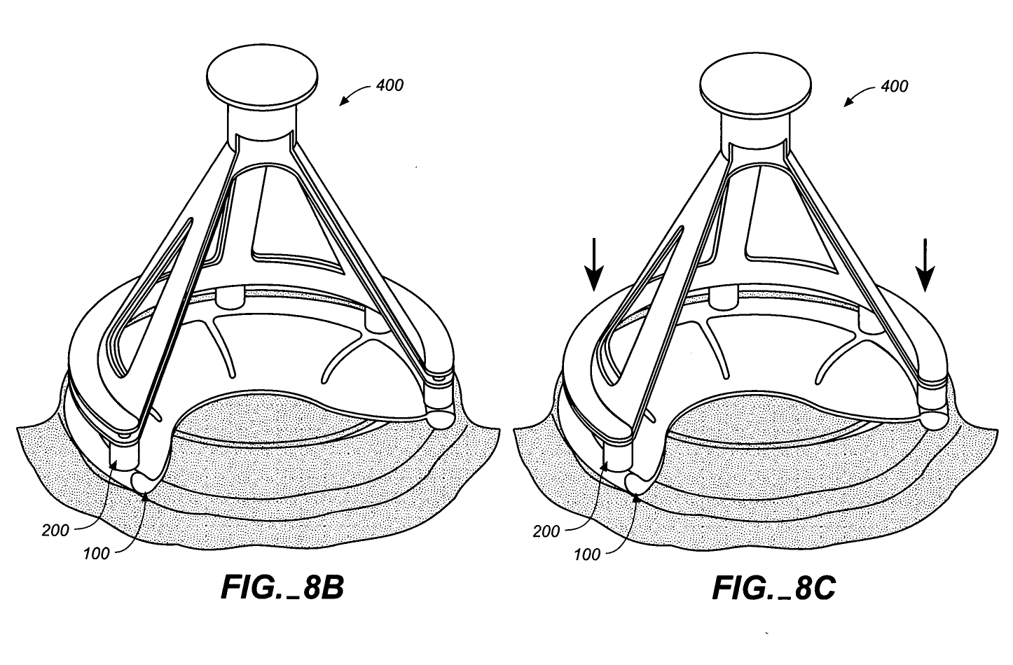

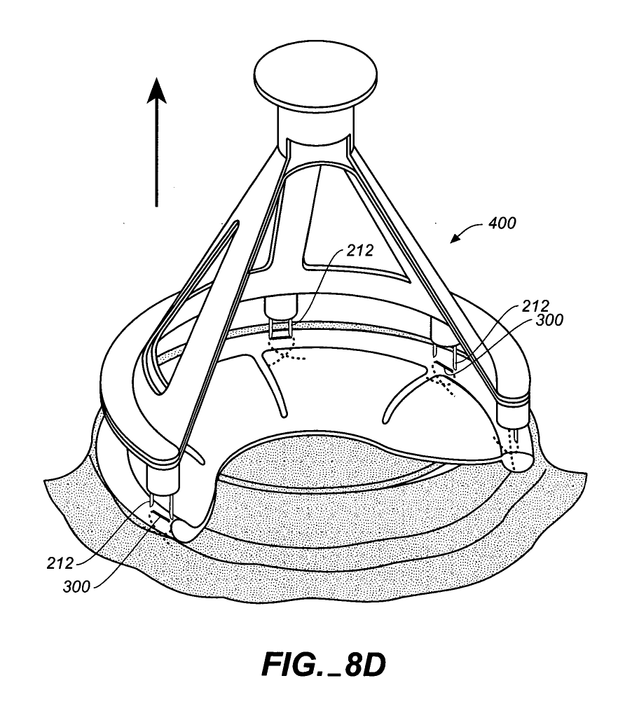

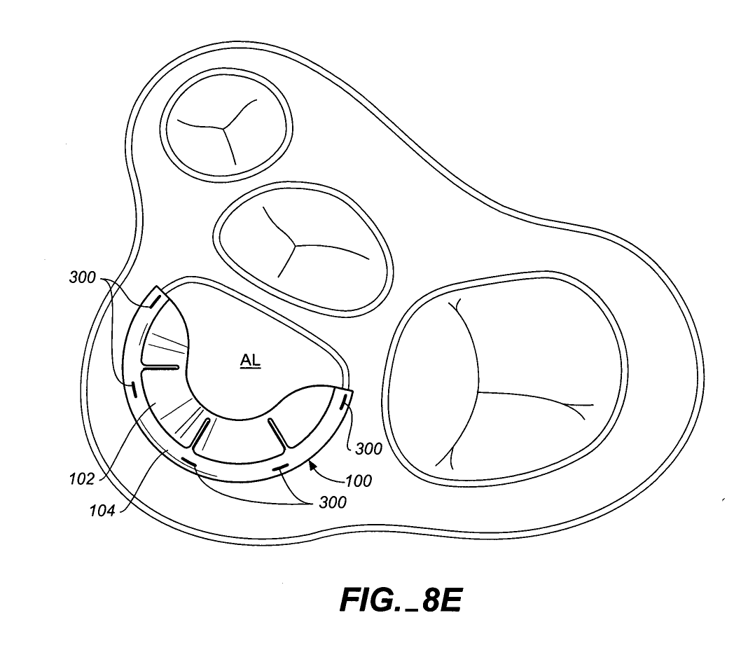

When clips 300 are positioned in clip delivery apparatus 200 in an open, deformed configuration as shown, for example, in FIGS. 4A and 5A, the clips maintain the ejectors in a proximal position or loaded position with rings 406 and 408 spaced from one another as shown, for example, in FIG. 7. Guide tubes 212 restrain the clips in the illustrated open configuration and the interaction of the restraining force of guide tubes 212 and the tendency of the clips to return to their relaxed state maintains the clip delivery apparatus in the position shown in FIGS. 4A and 4B and valve prosthesis delivery apparatus 400 in the position shown, for example, in FIGS. 7, 8A, and 8B until additional force is placed on ejector heads or anvils 210 (FIG. 8C). The materials used for valve prosthesis delivery apparatus 400 can include a combination of plastic and metal materials suitable for medical use. For example, clip delivery apparatus 200, ring 406 and anvil 408 can be medical grade stainless steel and the remaining components of delivery apparatus 400 can be plastic such as polyurethane or polycarbonate material. Alternatively, apparatus 200 can be stainless steel and the remaining components of apparatus 400 can be made of the foregoing plastic material.

Although particular configurations have been shown regarding first and second members 402 and 404 and the clip delivery support and anvil members, other configurations can be used without departing from the scope of the invention. For example, the clip delivery support and anvil members can be full rings.

The following example is set forth to illustrate operation of the invention, and is not intended to limit its scope. Referring to FIGS. 8A-8E, an exemplary method of using prosthesis 100 to treat mitral valve insufficiency is shown in accordance with the present invention.

As noted above, a competent mitral valve (MV) allows one-way flow of oxygenated blood that has entered the left atrium from the lungs to enter the left ventricle. The left ventricle then pumps the oxygenated blood to the rest of the body.

Referring to FIG. 8A, the mitral valve (MV) comprises a pair of leaflets, the anterior leaflet (AL) and the posterior leaflet (PL) of which the latter is larger. The base of each leaflet is attached to the mitral valve annulus (MVA). The mitral valve annulus includes a posterior portion (PP) and an anterior portion (AP) also known as the inter-trigone section, which is a generally straight substantially rigid section. The posterior portion of the annulus is a flexible, curved section that encompasses a larger portion of the annulus circumference than the anterior portion. The right and left fibrous trigones (generally indicated with reference characters RT and LT) mark the end of the generally straight section (inter-trigone section) and define the intersection points between the posterior and anterior portions (PP, AP).

The leaflets open and close in response to pressure differences on either side thereof. However, when the leaflets do not fully close, regurgitation and valve insufficiency can result. One method to treat the insufficiency using the implant or prosthetic apparatus of FIG. 1A will be described with reference to FIGS. 8B-8E.

A patient is placed on cardio-pulmonary bypass and prepared for open chest/open heart surgery, which typically requires a sternotomy. The surgeon opens the left atrium of the heart and measures the size and shape of the mitral valve annulus. A valve prosthesis 100 is selected based on the measured size and shape of the annulus so that ring or partial ring 104 will conform to the size and shape of the annulus. Accordingly, the size and shape of curved member 104 is selected to match the size and shape of that portion or all of the annulus upon which it is to be seated. The diameter of curved member 104 can range form about 18 mm to about 45 mm, and more typically will range from abut 24 mm to about 36 mm. In the case where a partial ring such as illustrative member 104 is used, the curved member is selected so that it is sized and configured for attachment to the posterior portion of the mitral valve annulus of the patient's heart. The curved member 104 can then minimize or prevent further dilation of the annulus, while the replacement leaflet 102 corrects the mitral regurgitation. In this manner, valve prosthesis 100 can simplify valve repair procedures.

The selected valve prosthesis is then aligned with the exposed ends of clips 300 of valve prosthesis delivery apparatus or mechanism 400 as shown in FIG. 7 and curved or leaflet support member 104 is pressed against the clips, while applying downward pressure to plunger 410 so that the piercing ends of the clips pass through leaflet support member 104 as shown in FIG. 8A. Alternatively, the clips may remain retracted until tubes 212 contact support member 104 after which time they are partially ejected to partially extend from the opposite side of leaflet support member as shown in FIG. 8A. With the prosthesis secured to the prosthesis delivery apparatus, the prosthesis delivery apparatus is seated on the valve annulus as shown in FIG. 8B. Plunger or knob 410 is then pressed downwardly to move first member 402 and ring 406 downardly as shown in FIG. 8C to effectuate simultaneous ejection of all of the clips from the clip delivery apparatus with a single stroke or actuation step. After the clips have been ejected into the mitral valve annulus, they move toward their closed configurations to secure the valve prosthesis to the mitral valve as shown in FIG. 8D after which the prosthesis delivery apparatus is removed. The implant and delivery apparatus can provide a quick and effective way to treat mitral valve regurgitation. The implant can be attached to the posterior portion of the mitral valve annulus such that the implant skirt or prosthetic leaflet coapts with the opposed natural leaflet and skirt support member 104 constructed to prevent further dilation of the annulus.

The implanted prosthesis shown in FIG. 8E, illustrates a top view of the valve prosthesis in place over the mitral valve with the anterior leaflet in view and in a closed position with the prosthetic leaflet or skirt covering the natural posterior leaflet. Although the natural posterior leaflet chordae tendineae remains in place so that it can still function, leaflet coaption now occurs between the natural anterior leaflet AL and the replacement posterior leaflet 102. If the natural posterior leaflet chordae tendineae were removed, the ventricle could sag or expand further over time, which would make it less efficient.

As noted above, the annuloplasty ring or member 102 can be constructed to strengthen the annulus and prevent any further distension of the annulus when secured thereto. Member 102 also can be used to shorten the annulus to treat eschemic mitral regurgitation as is done with annuloplasty rings. In this case, valve prosthesis member 100 would not be delivered with valve prosthesis apparatus 400. Rather, the portion of member 100 that is to be secured to the annulus would be delivered or secured to the annulus with sutures in a manner known in the art to shorten the annulus.

Although the foregoing method has been described in connection with open chest surgery, the prosthesis and delivery apparatus described herein can be used with minimally invasive approaches that typically require a thoracotomy between adjacent ribs.

Variations and modifications of the devices and methods disclosed herein will be readily apparent to persons skilled in the art. As such, it should be understood that the foregoing detailed description and the accompanying illustrations, are made for purposes of clarity and understanding, and are not intended to limit the scope of the invention, which is defined by the claims appended hereto.

1. Heart valve prosthesis for placement in a valve of a patient's heart, said valve prosthesis comprising a surgical implant including a curved member and a skirt, said curved member having first and second ends and being adapted to form a partial ring along a portion of one of the valve annulae in the patient's heart, and said skirt extending along said curved member and depending therefrom.

2. The prosthesis of claim 1 wherein said curved member is flexible.

3. The prosthesis of claim 1 wherein said curved member is rigid.

4. The prosthesis of claim 1 further including a plurality of struts extending radially inward from said curved member.

5. The prosthesis of claim 4 wherein said struts are integrally formed with said curved member.

6. The prosthesis of claim 4 wherein said skirt has an inner perimeter and said struts terminate before said inner perimeter.

7. The prosthesis of claim 1 wherein said skirt comprises prosthetic tissue.

8. The prosthesis of claim 1 wherein said skirt comprises ePTFE.

9. The prosthesis of claim 1 further including fibrous mesh surrounding said curved member.

10. Heart valve prosthesis for placement in a valve of a patient's heart, said valve prosthesis comprising a surgical implant including a closed ring shaped member and a skirt, said ring shaped member being adapted to form a ring along one of the valve annulae in the patient's heart, and said skirt extending along at least a portion of said ring shaped member and depending therefrom.

11. The prosthesis of claim 10 wherein said curved member is flexible.

12. The prosthesis of claim 10 wherein said curved member is rigid.

13. The prosthesis of claim 10 further including a plurality of struts extending radially inward from said ring shaped member.

14. The prosthesis of claim 13 wherein said struts are integrally formed with said ring shaped member.

15. The prosthesis of claim 13 wherein said skirt has an inner perimeter and said struts terminate before said inner perimeter.

16. The prosthesis of claim 10 wherein said skirt comprises prosthetic tissue.

17. The prosthesis of claim 10 wherein said skirt comprises ePTFE.

18. The prosthesis of claim 10 further including fibrous mesh surrounding said ring shaped member.

19. Heart valve delivery apparatus for placing heart valve prosthesis in a patient's heart, said apparatus comprising:
a delivery device comprising a plurality of tube pairs arranged to support said heart valve prosthesis; and
a plurality of self-closing clips, each clip having an open configuration and a closed configuration and first and second piercing ends, each clip being ejectably mounted to one of said tube pairs with a first portion of the clip slidably positioned in one tube of the tube pair and a second portion slidably positioned in the other tube of the tube pair so that the first clip piercing end can be ejected from said one tube of the tube pair and said second piercing end can be ejected from said other tube of the tube pair.

20. The apparatus of claim 19 further including a plunger, each of said clips being coupled to said plunger.

21. The apparatus of claim 20 wherein said clips are laterally spaced from one another and arranged for parallel ejection.

22 Heart valve repair apparatus for placing heart valve prosthesis in a patient's heart, said apparatus comprising:
heart valve prosthesis comprising a prosthetic valve leaflet and a member supporting said leaflet; and
delivery apparatus comprising a support for said valve prosthesis and a plurality of clips ejectably mounted to said delivery apparatus support, each clip having two piercing tips extending into said member supporting said leaflet.

23. The heart valve repair apparatus of claim 22 further including a plunger, each of said clips being coupled to said plunger.

24. The heart valve repair apparatus of claim 23 wherein said clips are laterally spaced from one another and arranged for parallel ejection.

25. The heart valve repair apparatus of claim 22 wherein said clips have an open configuration and a closed loop shaped configuration, said clips being in said open configuration.

26. The heart valve repair apparatus of claim 22 further including a plurality of tube pairs, each clip having a first portion slidably positioned in one tube of a tube pair and a second portion slidably positioned in the other tube of said tube pair.

27. A method for delivering heart valve prosthesis comprising:
providing heart valve prosthesis having a curved member and a skirt extending therefrom and a plurality of self-closing clips, each having two pointed ends, and an open configuration and a closed configuration;
securing the curved member to said plurality of self-closing clips with the two pointed ends of each clip penetrated into the curved member;
placing the curved member on the mitral valve annulus of a patient's heart;
ejecting all of the clips simultaneously to penetrate into the mitral valve annulus and move toward their closed configuration to secure the heart valve prosthesis to the valve annulus.

28. The method of claim 27 wherein the heart valve prosthesis has a plurality of struts extending radially inward from said curved member.

29. The method of claim 27 wherein said curved member forms a partial ring which is placed along the posterior portion of the mitral valve annulus with the skirt extending over the mitral valve posterior leaflet.

30. The method of claim 27 wherein said curved member forms a closed ring which is arranged so that the skirt extends over the mitral valve posterior leaflet.

31. The method of claim 27 wherein said curved member is flexible.

32. The method of claim 27 wherein said curved member is rigid.
